(19) United States
(12) Patent Application Publication
(10) Pub. No.: US 2014/0194363 A1
(43) Pub. Date: Jul. 10, 2014

(54) Compression-resistant collagen-based artificial bone repair material

(71) Applicant: Eric Gang Hu, Basking Ridge, NJ (US)

(72) Inventor: Eric Gang Hu, Basking Ridge, NJ (US)

(73) Assignee: Beijing Allgens Medical and Technology Co, Ltd, Beijing (CN)

(21) Appl. No.: 13/736,371

(22) Filed: Jan. 8, 2013

(51) Int. Cl. A61K 38/39

(52) U.S. Cl. CPC A61K 38/39
USPC 514/16.7

(57) ABSTRACT

The present invention provides a compression-resistant collagen-based artificial bone repair material that could be used for bone defect repair at human load-bearing sites. Such material has a composition and structure of self-assembled nano-sized calcium phosphate salt and collagen molecules, thereby possessing a biomimetic mineralization structure similar to the natural bone. On the aspect of mechanical properties, such material has a similar mechanical strength to human cortical bone, which makes it suitable for repairing bone defects at human load-bearing sites. The present invention further provides preparation methods of such Compression-resistant collagen-based artificial bone repair material.

### FIELD OF THE INVENTION

The present invention relates to the field of biomedical materials, and more specific, to a compression-resistant collagen-based artificial bone repair material for load-bearing bone defect repairing, as well as the methods for preparing the same.

### BACKGROUND OF THE INVENTION

Bone defects caused by trauma, infection and bone tumor at load-bearing sites are common orthopedic diseases. Many kinds of Ti-alloy, polyether ether ketone and carbon fiber materials have been developed for repairing such bone defects. However, each of these materials has its own drawbacks in terms of mechanical properties and bioactivities. Elastic modulus of Ti-alloy is 4 to 10 times compared to that of human bone[1], thus leading to stress shielding in clinical applications that adverse to bone repair. Carbon fiber is easy to produce wear debris on the surface [2], which would trigger inflammatory response at the repair site, so as to affect repair effect. Moreover, the biocompatibilities of Ti-alloy, polyether ether ketone and carbon fiber are derived from their bioinertness [3,4]. These biomaterials do not possess bioactivity that facilitate osteointegration to human bone, so they are primarily used as permanent implant, without involving in metabolism, thus resulting in unsatisfactory results for long-term bone repair.

Natural human bone tissue is mainly composed of collagen and hydroxyapatite as the main organic and inorganic component respectively [5]. Wherein, the collagen has regular hierarchitecture and provides template for biomineralization of hydroxyapatite, so as to form well-organized mineralized collagen composite. Bioactive artificial bone repair material that mimics both component and structure of natural bone is able to provide a microenvironment similar to those of natural bone. Such microenvironment is helpful to attachment and proliferation of osteocytes, thereby promoting bone defect repair [6].

A number of hard tissue repair products made of collagen/hydroxyapatite as the main component have been developed in recent years, such as guided tissue regeneration (GTR) membranes. For these products, the ratio of collagen to hydroxyapatite is 2/8˜7/3, which could promote bone tissue regeneration [7]. However, such GTR membranes are mainly used for repairing non-load-bearing sites, such as filling periodontal defects and spinal intertransverse fusion [8]. Because of their flexibility, the GTR membranes are unable to provide mechanical support for the repairing sites. Besides, most of the existing collagen/hydroxyapatite composite bone repair materials are physical mixtures of collagen and hydroxyapatite, rather than the hierarchitecture of the natural bone.

To meet the mechanical requirement of the materials used for bone repair, calcium phosphate bioceramics represented by hydroxyapatite have been widely investigated, and this kind of scaffolds possessing certain mechanical strength have been developed, for example, a porous hydroxyapatite bioceramic scaffold [9]. However, the indispensable high-temperature sintering process in the preparation of calcium phosphate ceramics limits the composition that some organic components beneficial to bone repair (e.g. collagen) cannot be incorporated in the final product. The sintered calcium phosphate ceramics are too fragile to be used as bone repair material at the load-bearing sites. Otherwise, it is reported by literatures that nano-sized hydroxyapatite is more favorable to osteogenic differentiation of cells than the microparticles [10]. Even the raw material is nano-sized hydroxyapatite particles (particle size<100 nm), the crystalline grains will grow to micron-size during the sintering, thereby decreasing osteogenic activity of the material.

As a result, prior arts and products cannot provide bioactive materials for bone repair at human load-bearing sites.

### REFERENCES

[1] Huiskes R, Weinans H, van Rietbergen B. The relationship between stress shielding and bone resorption around total hip stems and the effects of flexible materials. Clin Orthop Relat Res. 1992, 274: 124-134.
[2] Howling G I, Sakoda H, Antonarulrajah A, et al. Biological response to wear debris generated in carbon based composites as potential bearing surfaces for artificial hip joints. J Biomed Mater Res B Appl Biomater, 2003, 67(2): 758-764.
[3] Nebe J B, Müller L, Lüthen F, et al. Osteoblast response to biomimetically altered titanium surfaces. Acta Biomater, 2008, 4(6): 1985-1995.
[4] Zhao M, An M, Wang Q, et al. Quantitative proteomic analysis of human osteoblast-like MG-63 cells in response to bioinert implant material titanium and polyetheretherketone. J Proteomics, 2012, 75(12): 3560-3573.
[5] Olszta M J, Cheng X, Jee S S, et al. Bone structure and formation: a new perspective. Mater Sci Eng R-Rep, 2007, 58: 77-116.
[6] Liao S S, Cui F Z, Zhang W, et al. Hierarchically biomimetic bone scaffold materials: Nano-HA/collagen/PLA composite. J Biomed Mater Res B Appl Biomater, 2004, 69B(2): 158-165.
[7] Oortgiesen D A, Plachokova A S, Geenen C, et al. Alkaline phosphatase immobilization onto Bio-Gide® and Bio-Oss® for periodontal and bone regeneration. J Clin Periodontol, 2012, 39(6): 546-555.
[8] Bottino M C, Thomas V, Schmidt G, et al. Recent advances in the development of GTR/GBR membranes for periodontal regeneration—a materials perspective. Dent Mater. 2012, 28(7): 703-721.
[9] Fan X, Case E D, Ren F, et al. Part II: fracture strength and elastic modulus as a function of porosity for hydroxyapatite and other brittle materials. J Mech Behav Biomed Mater, 2012, 8: 99-110.
[10] Huang Y, Zhou U, Zheng L, et al. Micro-/nano-sized hydroxyapatite directs differentiation of rat bone marrow derived mesenchymal stem cells towards an osteoblast lineage. Nanoscale, 2012, 4(7): 2484-2490.

### SUMMARY OF THE INVENTION

The primary objective of this invention is to provide a compression-resistant collagen-based artificial bone repair material. Such material has a composition and structure of self-assembled nano-sized calcium phosphate salt (nano-Ca-P) and collagen molecules, thereby possessing a biomimetic mineralization structure similar to the natural bone. On the aspect of mechanical properties, such material has a similar mechanical strength to human cortical bone, which make it suitable for repairing bone defects at human load-bearing sites. The present invention further provides preparation methods of such Compression-resistant collagen-based artificial bone repair material.

The present invention provides a compression-resistant collagen-based artificial bone repair material. The material is a dense and homogeneous organic/inorganic composite material. The organic phase contains collagen, and could also contain polyester, which contains one or more of the following: poly (lactic acid) (PLA), poly (glycolic acid) (PGA), poly (lactic-co-glycolic acid) (PLGA), polycaprolactone (PCL), polydiaxane (PDO) and the co-polymer of the above; the inorganic phase contains nano-sized calcium phosphate salt. The weight ratio of the organic phase to the inorganic phase is 9/1˜4/6 in the organic/inorganic composite. When the polyester is incorporated, the weight ratio of collagen to polyester is 9/1˜1/9 in the organic phase.

Said compression-resistant collagen-based artificial bone repair material possesses a compressive strength of 65˜150 MPa and a bending strength of 20˜100 MPa.

Said nano-sized calcium phosphate salt possesses a particle size of 20˜200 nm, and a molar ratio of Ca/P=1/1˜2/1. Advantageously, said nano-sized calcium phosphate salt is nano-sized hydroxyapatite with a particle size of 20˜200 nm

Said polyester possesses a molecular weight of 50,000˜500,000.

The present invention further provides a preparation method of said compression-resistant collagen-based artificial bone repair material as the first aspect of this invention is provided, comprising:

Step S1. Preparation of nano-Ca-P/collagen biomimetic composite powders, further comprising:

Step S1-1. Dissolve collagen in any one of hydrochloric acid, nitric acid or acetic acid to form an acidic collagen solution, wherein, the concentration of the collagen is 5.0×10⁻⁵˜5.0×10⁻³ g/mL;

Step S1-2. Keep stiffing the solution obtained by step S1-1 and add Ca²⁺ containing solution dropwise, wherein, the addition of Ca²⁺ is 0.01˜0.16 mol for 1 g of collagen;

Step S1-3. Keep stiffing the solution obtained by step S1-2 and add PO₄³⁻ containing solution dropwise, wherein, the molar ratio of the added PO₄³⁻ and the added Ca²⁺ in S1-2 is Ca/P=1/1˜2/1;

Step S1-4. Keep stiffing the solution obtained by step S1-3 and add NaOH solution until the pH of the mixture system gets to 6˜8, wherein, precipitation appears when the pH of the mixture system gets to 5˜6, and white suspension will be obtained when the pH gets to 7;

Step S1-5. Stand the mixture system obtained by step S1-4 for 24˜120 hours, and then separate out the precipitation and wash it to remove impurity ions, followed by a freeze-drying, the composite powders will be obtained after gridding.

Step S2. Preparation of collagen/nano-Ca-P/polyester composite, further comprising:

Step S2-1. Preparation polyester solution with a concentration of 0.02˜0.15 g/mL by dissolving the polyester with molecular weight of 50,000˜500,000 into any one of 1,4-dioxane, dichloromethane, chloroform or dimethyl sulfoxide at 40˜70° C., said polyester could be one or more of PLA, PGA, PLGA, PCL, PDO, or the copolymers;

Step S2-2. Add the composite powders obtained by step S1-5 into the polyester solution obtained by step S2-1 to form a collagen/nano-Ca-P/polyester mixture suspension system, wherein, the weight ratio of the composite powders to the polyester within the polyester solution is 1/2˜3/2;

Step S2-3. Put the mixture suspension system obtained by step S2-2 into an environment of −20˜4° C. to thoroughly freeze, and/or into liquid nitrogen to deep freeze, and then freeze-dry for 24˜72 hours, followed by transferring to a vacuum drying oven to dry for 72˜120 hours, thus obtaining a collagen/nano-Ca-P/polyester composite;

Step S2-4. Smash the composite obtained by step S2-3 and sieve to screen out composite powders with particle size of 100˜600 μm.

Step S3. Cold compression molding of the composite, further comprising:

Step S3-1. Weigh the composite powders obtained by step S2-4 and fill the powders into a cold compression dies;

Step S3-2. Compress the dies and make the pressure applied to the composite powders reaches 200˜1400 MPa;

Step S3-3. Keep the pressure for 30˜300 seconds, and then demould to obtain the Compression-resistant collagen-based artificial bone repair material.

Above operation procedures is used for preparing collagen/nano-Ca-P/polyester three-phase compression-resistant collagen-based artificial bone repair material. If the compression-resistant collagen-based artificial bone repair material is only consisted of collagen and nano-Ca-P without polyester, the preparation process would skip the step S2, and the step S3-1 would be modified to “weigh the composite powders obtained by step S1-5 and fill the powders into a cold compression dies”. The rest steps remain unchanged.

The present invention provides a compression-resistant collagen-based artificial bone repair material. The material is a dense-porous bi-layer organic/inorganic composite, wherein, the organic phase contains both collagen and polyester, and the inorganic phase contains nano-sized calcium phosphate salt. Said polyester could be one or more of PLA, PGA, PLGA and PCL. The weight ratio of the organic phase to the inorganic phase is 9/1˜2/8, and the weight ratio of collagen to polyester is 9/1˜1/9.

Said bi-layer structure is provided with

a dense layer as the lower layer, with a thickness of 0.5˜5 mm, a compressive strength of 65˜150 MPa and a bending strength of 20˜100 MPa; and

a porous layer as the upper layer, with a thickness of 0.5˜5 mm and a porosity of 50%˜80%.

Said nano-sized calcium phosphate salt possesses a particle size of 20˜200 nm, and a molar ratio of Ca/P=1/1˜2/1. Advantageously, said nano-sized calcium phosphate salt is nano-sized hydroxyapatite with particle size of 20˜200 nm

Said polyester possesses a molecular weight of 50,000˜500,000.

The present invention provides a preparation method of said dense-porous bi-layer compression-resistant collagen-based artificial bone repair material as the third aspect of this invention is provided, comprising:

Step S 1. Preparation of nano-Ca-P/collagen biomimetic composite powders, further comprising:

Step S1-1. Dissolve collagen in any one of hydrochloric acid, nitric acid or acetic acid to form an acidic collagen solution, wherein, the concentration of the collagen is 5.0×10⁻⁵˜5.0×10⁻³ g/mL;

Step S1-2. Keep stiffing the solution obtained by step S1-1 and add Ca²⁺ containing solution dropwise, wherein, the addition of Ca²⁺ is 0.01˜0.16 mol for 1 g of collagen;

Step S1-3. Keep stiffing the solution obtained by step S1-2 and add PO₄³⁻ containing solution dropwise, wherein, the molar ratio of the added PO₄³⁻ and the added Ca²⁺ in S1-2 is Ca/P=1/1˜2/1;

Step S1-4. Keep stiffing the solution obtained by step S1-3 and add NaOH solution until the pH of the mixture system gets to 6˜8, wherein, precipitation appears when the pH of the mixture system gets to 5˜6, and white suspension will be obtained when the pH gets to 7;

Step S1-5. Stand the mixture system obtained by step S1-4 for 24˜120 hours, and then separate out the precipitation and wash it to remove impurity ions, followed by a freeze-drying, the composite powders will be obtained after gridding.

Step S2. Preparation of collagen/nano-Ca-P/polyester composite, further comprising:

Step S2-1. Preparation polyester solution with a concentration of 0.02˜0.15 g/mL by dissolving polyester with molecular weight of 50,000˜500,000 into any one of 1,4-dioxane, dichloromethane, chloroform or dimethyl sulfoxide at 40˜70° C., said polyester could be one or more of PLA, PGA, PLGA, PCL, PDO or the copolymers of them;

Step S2-2. Add the composite powders obtained by step S1-5 into the polyester solution obtained by step S2-1 to form a collagen/nano-Ca-P/polyeter mixture suspension system, wherein, the weight ratio of the composite powders to the polyester within the polyester solution is 1/2˜3/2;

Step S2-3. Put the mixture suspension system obtained by step S2-2 into an environment of −20˜4° C. to thoroughly freeze, and/or into liquid nitrogen to deep freeze, and then freeze-dry for 24˜72 hours, followed by transferring to a vacuum drying oven to dry for 72˜120 hours, thus obtaining a collagen/nano-Ca-P/polyester composite;

Step S2-4. Smash the composite obtained by step S2-3 and sieve to screen out composite powders with particle size of 100˜600 μm.

Step S3. Cold compression molding of the composite, further comprising:

Step S3-1. Weigh the composite powders obtained by step S2-4 and fill the powders into a cold compression dies;

Step S3-2. Compress the dies and make the pressure applied to the composite powders reaches 200˜1400 MPa;

Step S3-3. Keep the pressure for 30˜300 seconds, and then demould to obtain said dense layer.

Step S4. Fabrication of porous layer on the dense layer, further comprising:

Step S4-1. Use the dense layer obtained by step S3-3 as the substrate, and repeat steps S1-1˜S2-2 to cover the collagen/nano-Ca-P/polyester mixture suspension obtained by step S2-2 on such substrate, then standing for 2˜15 min, meanwhile slight solvation occurs on the substrate upper surface;

Step S4-2. Put the dense and the covered mixture suspension obtained by step S4-1 into a low-temperature environment of −20˜−10° C. to achieve quick-freezing, freeze-dry them for 24˜72 hours, and then transfer to a vacuum drying oven to dry for 72˜120 hours, thus finally obtain said dense-porous bi-layer Compression-resistant collagen-based artificial bone repair material.

By implementing the present invention, compression-resistant collagen-based artificial bone repair material with a similar mechanical strength to human cortical bone could be prepared, so as to meet the clinical requirement of bone repair at load-bearing sites. The material contains main component of human natural bone, such as collagen and nano-Ca-P, as well as collagen and nano-Ca-P form biomimetic mineralization structure similar to the natural bone via self-assembly, thus providing excellent microenvironment for attachment and proliferation of osteocytes in terms of composition and structure. As a result, the compression-resistant collagen-based artificial bone repair material provided by the present invention has good bioactivity and excellent mechanical properties, and is biodegradable as well. The current material fills in the gaps of Compression-resistant bioactive bone repair materials demanded by clinics, thus possessing promising application prospect.

### BRIEF DESCRIPTION OF THE DRAWINGS

FIG. 1 shows a process flow chart for preparing the compression-resistant collagen-based artificial bone repair material, according to the second aspect of the present invention;

FIGS. 2A and 2B show a schematic diagram of a compression-resistant collagen-based artificial bone repair material used for human spinal fusion, according to the first aspect of the present invention, wherein, FIG. 2A is the front view and FIG. 2B is the lateral view;

FIG. 3 shows a schematic diagram of a compression-resistant collagen-based artificial bone repair material used for human vertebral plate repairing, according to the first aspect of the present invention;

FIG. 4 shows a schematic diagram of a dense-porous bi-layer compression-resistant collagen-based artificial bone repair material, according to the third aspect of the present invention;

FIG. 5 shows a process flow chart for preparing the dense-porous bi-layer compression-resistant collagen-based artificial bone repair material, according to the forth aspect of the present invention.

### DETAILED DESCRIPTION OF THE PREFERRED EMBODIMENT

Further detail of the present invention will become evident from the attached drawings and operation manners.

1. Preparation of a Compression-Resistant Collagen/Nano-Ca-P Artificial Bone

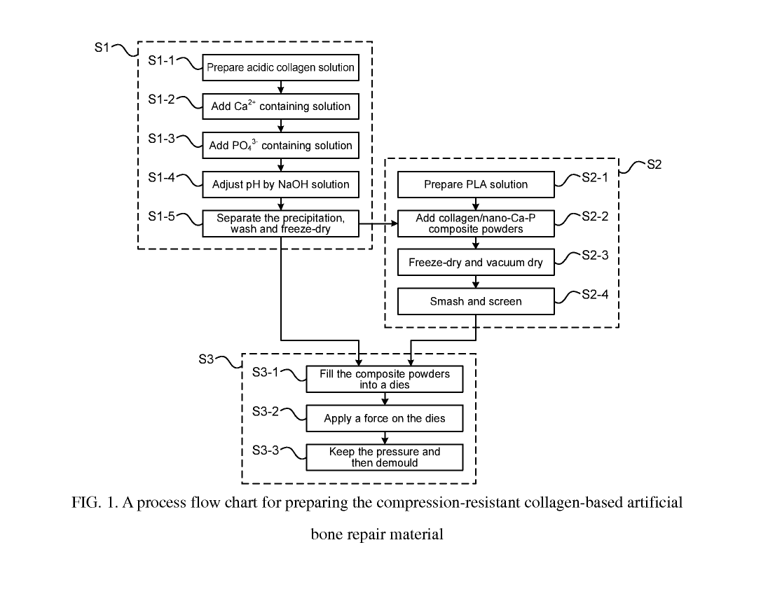

FIG. 1 shows a process flow chart for preparing the compression-resistant collagen-based artificial bone repair material of the present invention. According to the steps shown in FIG. 1, a method for preparing this compression-resistant collagen/nano-Ca-P artificial bone is:

Step S1-1. Dissolve 1 g of collagen in 2 L of 0.5 mol/L acetic acid solution to prepare an acidic collagen solution;

Step S1-2. Keep stiffing the solution obtained by step S1-1 and add 100 mL of 1 mol/L CaCl₂ solution dropwise;

Step S1-3. Keep stirring the solution obtained by step S1-2 and add 100 mL of 0.6 mol/L Na₂HPO₄ solution dropwise;

Step S1-4. Keep stiffing the solution obtained by step S1-3 and add 1 mol/L NaOH solution until the pH of the mixture system gets to 7;

Step S1-5. Stand the mixture system obtained by step S1-4 for 48 hours, and then separate out the precipitation and wash it 5 times by centrifugation, followed by a freeze-drying, the composite powders will be obtained after gridding.

Because polyester is not involved, this embodiment skips step S2 and directly proceed to step S3.

Step S3-1. Weigh 0.6 g of the composite powders obtained by step S1-5 and fill the powders into a dies with the diameter of 12 mm;

Step S3-2. Apply a force of 75 kN on the dies;

Step S3-3. Keep the pressure for 90 seconds, and then de-mould to obtain the compression-resistant collagen/nano-Ca-P artificial bone.

The shape of this artificial bone is a disc with Φ=12 mm and h=3 mm. The test results show that the composite artificial bone has a density of 1.77 g/cm³, a compressive strength of 98 MPa and a bending strength of 32 MPa, thereby attaining the level of human cortical bone and being suitable for repairing human cortical bone defects.

2. Preparation of a Compression-Resistant Collagen/Nano-Ca-P/PLA Composite Artificial Bone

Firstly, prepare collagen/nano-Ca-P composite according to steps S1-1˜S1-5 of the embodiment 1.

Then, prepare collagen/nano-Ca-P/PLA composite, further comprising:

Step S2-1. Weigh 1 g of PLA (MW=100,000) and dissolve it in 10 mL of 1,4-dioxane at 50° C. to prepare a PLA solution;

Step S2-2. Add 1 g of the composite powders obtained by step S1-5 into the PLA solution obtained by step S2-1 to form a collagen/nano-Ca-P/PLA mixture suspension system;

Step S2-3. Put the mixture suspension system obtained by step S2-2 into an environment of −10° C. for 2 hours, and then freeze-dry for 48 hours, followed by transferring to a vacuum drying oven with a vacuum degree of −0.1 MPa to dry for 72 hours, thus obtaining a collagen/nano-Ca-P/PLA composite;

Step S2-4. Smash the composite obtained by step S2-3 and sieve to screen out composite powders with particle size of 100˜500 μm.

Finally, perform steps S3-1˜S3-3 of embodiment 1. The pressure applied on the dies of step S3-2 is adjusted to 120 kN, and other operations and parameters remain unchanged. After that, the artificial collagen/nano-Ca-P/PLA composite artificial bone is obtained.

By testing, such composite artificial bone has a density of 1.85 g/cm³, a compressive strength of 129 MPa, and a bending strength of 62 MPa. This artificial bone is suitable for repairing human bone defects at load-bearing site.

3. Preparation of a Compression-Resistant Collagen/Nano-Ca-P/PLGA Composite Artificial Bone

Prepare the composite according to steps of embodiment 2. Wherein, said PLA in step S2-1 is replaced by PLGA (LA/GA=50/50) with molecular weight of 120,000, and the applied pressure in step S3-2 is 155 kN. Other operations and parameters remain unchanged. Then, the artificial collagen/nano-Ca-P/PLGA composite artificial bone is obtained.

By testing, such composite artificial bone has a density of 1.85 g/cm³, a compressive strength of 145 MPa, and a bending strength of 84 MPa. This artificial bone is suitable for repairing human bone defects at load-bearing site.

4. Preparation of a Compression-Resistant Collagen/Nano-Ca-P/PCL Composite Spinal Fusion Cage

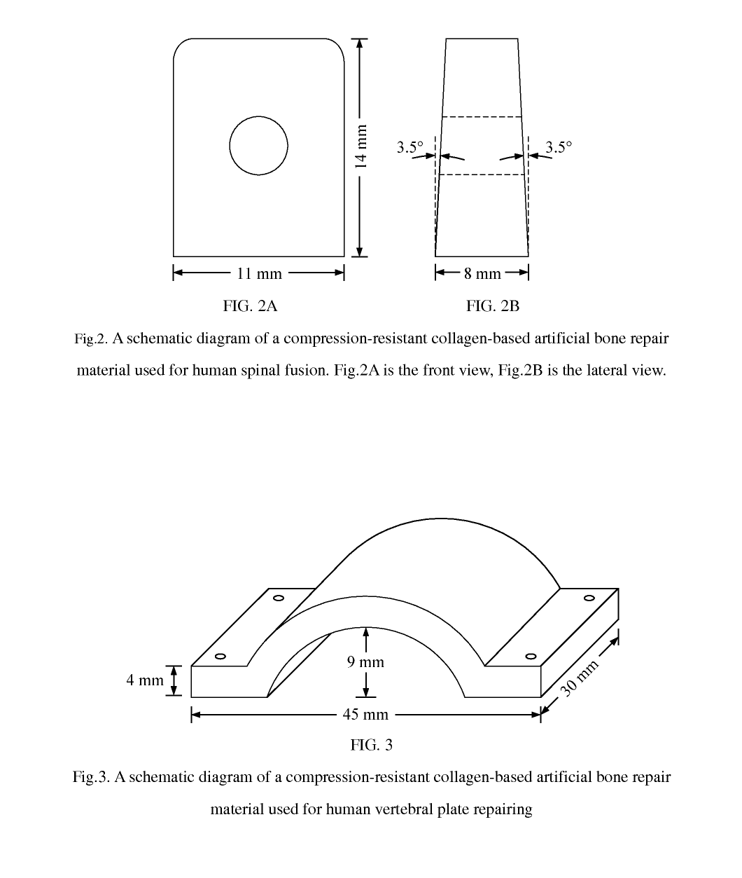

FIGS. 2A and 2B show a schematic diagram of a Compression-resistant collagen-based artificial bone repair material used for human spinal fusion of the present invention, wherein, FIG. 2A is the front view and FIG. 2B is the lateral view. Particularly, this artificial bone repair material is a collagen/nano-Ca-P/PCL composite spinal fusion cage. According to the steps shown in FIG. 1, a method for preparing this spinal fusion cage is:

Step S1-1. Dissolve 1 g of collagen in 1 L of 0.1 mol/L HCl solution to prepare an acidic collagen solution;

Step S1-2. Keep stiffing the solution obtained by step S1-1 and add 150 mL of 1 mol/L CaCl₂ solution dropwise;

Step S1-3. Keep stiffing the solution obtained by step S1-2 and add 100 mL of 1 mol/L H₃PO₄ solution dropwise;

Step S1-4. Keep stirring the solution obtained by step S1-3 and add 0.5 mol/L NaOH solution until the pH of the mixture system gets to 7;

Step S1-5. Stand the mixture system obtained by step S1-4 for 72 hours, and then separate out the precipitation and wash it 5 times by vacuum filtration, followed by a freeze-drying, the composite powders will be obtained after gridding.

Step S2-1. Weigh 1 g of PCL (MW=100,000) and dissolve it in 10 mL of dichloromethane at 50° C. to prepare a PCL solution;

Step S2-2. Add 1 g of the composite powders obtained by step S1-5 into the PCL solution obtained by step S2-1 to form a collagen/nano-Ca-P/PCL mixture suspension system;

Step S2-3. Put the mixture suspension system obtained by step S2-2 into an environment of −10° C. for 2 hours and then liquid nitrogen to deep freeze, freeze-dry for 48 hours, followed by transferring to a vacuum drying oven with a vacuum degree of −0.1 MPa to dry for 72 hours, thus obtaining a collagen/nano-Ca-P/PCL composite;

Step S2-4. Smash the composite obtained by step S2-3 and sieve to screen out composite powders with particle size of 100˜500 μm;

Step S3-1. Weigh 1.66 g of the composite powders obtained by step S2-4 and fill the powders into a dies for fabricating spinal fusion cage by pressing;

Step S3-2. Apply a force of 120 kN on the dies;

Step S3-3. Keep the pressure for 240 seconds, and then demould to obtain the Compression-resistant collagen/nano-Ca-P/PCL composite spinal fusion cage.

The test results show that the spinal fusion cage has a density of 1.8 g/cm³ and a compressive strength of 106 MPa, thereby meeting the clinical requirement of spinal fusion materials.

5. Preparation of a Compression-Resistant Collagen/Nano-Ca-P/PGA Composite Artificial Vertebral Plate

FIG. 3 shows a schematic diagram of a Compression-resistant collagen-based artificial bone repair material used for human vertebral plate repairing of the present invention. Particularly, this artificial bone repair material is a collagen/nano-Ca-P/PGA composite artificial vertebral plate. According to the steps shown in FIG. 1, a method for preparing this artificial vertebral plate is:

Step S1-1. Dissolve 6 g of collagen in 3 L of 0.1 mol/L HNO₃ solution to prepare an acidic collagen solution;

Step S1-2. Keep stiffing the solution obtained by step S1-1 and add 750 mL of 1 mol/L Ca(NO₃)₂ solution dropwise;

Step S1-3. Keep stiffing the solution obtained by step S1-2 and add 750 mL of 1 mol/L (NH₄)₂HPO₄ solution dropwise;

Step S1-4. Keep stirring the solution obtained by step S1-3 and add 0.5 mol/L NaOH solution until the pH of the mixture system gets to 7;

Step S1-5. Stand the mixture system obtained by step S1-4 for 96 hours, and then separate out the precipitation and wash it 5 times by vacuum filtration, followed by a freeze-drying, the composite powders will be obtained after gridding.

Step S2-1. Weigh 9 g of PGA (MW=150,000) and dissolve it in 200 mL of chloroform at 65° C. to prepare a PGA solution;

Step S2-2. Add 6 g of the composite powders obtained by step S1-5 into the PGA solution obtained by step S2-1 to form a collagen/nano-Ca-P/PGA mixture suspension system;

Step S2-3. Put the mixture suspension system obtained by step S2-2 into an environment of 0° C. for 2 hours and then liquid nitrogen to deep freeze, freeze-dry for 72 hours, followed by transferring to a vacuum drying oven with a vacuum degree of −0.1 MPa to dry for 96 hours, thus obtaining a collagen/nano-Ca-P/PGA composite;

Step S2-4. Smash the composite obtained by step S2-3 and sieve to screen out composite powders with particle size of 200˜500 μm.

Step S3-1. Weigh 11 g of the composite powders obtained by step S2-4 and fill the powders into a dies for fabricating artificial vertebral plate by pressing;

Step S3-2. Apply a force of 900 kN on the dies;

Step S3-3. Keep the pressure for 270 seconds, and then demould to obtain the Compression-resistant collagen/nano-Ca-P/PGA composite artificial vertebral plate.

The prepared artificial vertebral plate has a span of 45 mm, a length of 30 mm, a thickness of 4 mm and a height of 9 mm. The test results show that the artificial vertebral plate has a density of 1.74 g/cm³, a compressive strength of 98 MPa and a bending strength of 36 MPa, thereby meeting the clinical requirement of vertebral plate repair.

6. Preparation of a Dense-Porous Bi-Layer Compression-Resistant Collagen/Nano-Ca-P/PLA Composite Bone Repair Material

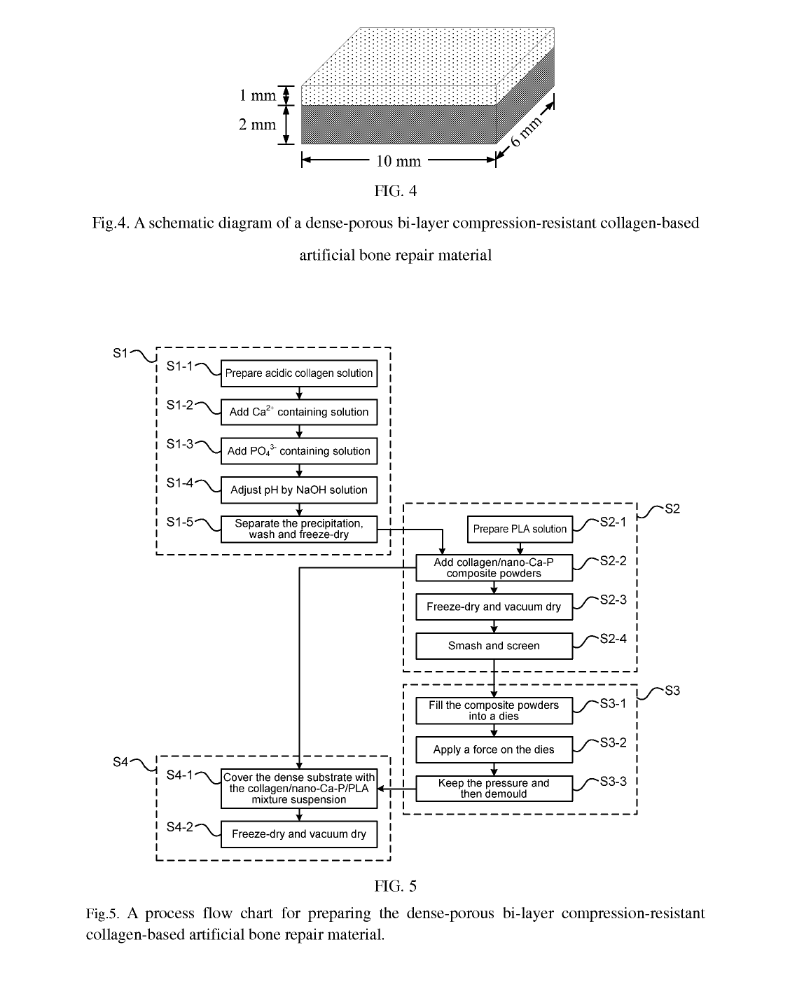

FIG. 4 shows a schematic diagram of a dense-porous bi-layer Compression-resistant collagen-based artificial bone repair material of the present invention, wherein, the lower layer is a dense layer with a thickness of 2 mm and the upper layer is a porous layer with a thickness of 1 mm. FIG. 5 shows a process flow chart for preparing the dense-porous bi-layer Compression-resistant collagen-based artificial bone repair material of the present invention. According to the steps shown in FIG. 5, a method for preparing the artificial bone repair material shown in FIG. 4 is:

Step S1-1. Dissolve 0.3 g of collagen in 3 L of 0.2 mol/L CH₃COOH solution to prepare an acidic collagen solution;

Step S1-2. Keep stirring the solution obtained by step S1-1 and add 100 mL of 0.15 mol/L CaCl₂ solution dropwise;

Step S1-3. Keep stirring the solution obtained by step S1-2 and add 60 mL of 0.15 mol/L (NH₄)₂HPO₄ solution dropwise;

Step S1-4. Keep stirring the solution obtained by step S1-3 and add 0.2 mol/L NaOH solution until the pH of the mixture system gets to 7;

Step S1-5. Stand the mixture system obtained by step S1-4 for 36 hours, and then separate out the precipitation and wash it 5 times by centrifugation, followed by a freeze-drying, the composite powders will be obtained after gridding.

Step S2-1. Weigh 0.2 g of PLA (MW=50,000) and dissolve it in 1.5 mL of dimethyl sulfoxide at 60° C. to prepare a PLA solution;

Step S2-2. Add 0.25 g of the composite powders obtained by step S1-5 into the PLA solution obtained by step S2-1 to form a collagen/nano-Ca-P/PLA mixture suspension system;

Step S2-3. Put the mixture suspension system obtained by step S2-2 into an environment of 0° C. for 2 hours, and then freeze-dry for 24 hours, followed by transferring to a vacuum drying oven with a vacuum degree of −0.1 MPa to dry for 72 hours, thus obtaining a collagen/nano-Ca-P/PLA composite;

Step S2-4. Smash the composite obtained by step S2-3 and sieve to screen out composite powders with particle size of 100˜500 μm.

Step S3-1. Weigh 0.22 g of the composite powders obtained by step S2-4 and fill the powders into a dies with a length of 10 mm and a width of 6 mm;

Step S3-2. Apply a force of 25 kN on the dies;

Step S3-3. Keep the pressure for 240 seconds, and then demould to obtain the dense layer;

Step S4-1. Use the dense layer obtained by step S3-3 as the substrate, repeat steps S1-1˜S2-2, and move 70 μL of collagen/nano-Ca-P/PLA mixture suspension obtained by step S2-2 to cover on such substrate, and then standing for 10 minutes, meanwhile slight solvation occurs on the substrate upper surface;

Step S4-2. Put the dense and the covered mixture suspension obtained by step S4-1 into a low-temperature environment of −20° C. to achieve quick-freezing, freeze-dry them for 36 hours, and then transfer to a vacuum drying oven to dry for 96 hours, thus finally obtain dense-porous bi-layer Compression-resistant collagen-based artificial bone repair material.

The prepared dense-porous bi-layer Compression-resistant collagen-based artificial bone repair material has a length of 10 mm, a width of 6 mm and a total height of 3 mm. The test results show that the density of the dense layer is 1.83 g/cm³ and that of the porous layer is 0.35 g/cm³; the bi-layer composite artificial bone repair material has a compressive strength of 81 MPa and a bending strength of 30 MPa, thereby being suitable for the clinical requirement of repairing cortical-cancellous bone complex defect.

Comparison 1

Prepare composite artificial bone according to embodiment 1, wherein, the force applied in step S3-2 is 15 kN with other process parameters remain unchanged.

Comparison 2

Prepare composite artificial bone according to embodiment 1, wherein, the force applied in step S3-2 is 170 kN with other process parameters remain unchanged.

Comparison 3

Prepare composite artificial bone according to embodiment 1, wherein, perform the step S3-2 without keeping the pressure any more, and other process parameters remain unchanged.

Comparison 4

Prepare composite artificial bone according to embodiment 2, wherein, the force applied in step S3-2 is 15 kN with other process parameters remain unchanged.

Comparison 5

Prepare composite artificial bone according to embodiment 2, wherein, the force applied in step S3-2 is 170 kN with other process parameters remain unchanged.

Comparison 6

Prepare composite artificial bone according to embodiment 3, wherein, the force applied in step S3-2 is 10 kN with other process parameters remain unchanged.

Comparison 7

Prepare composite artificial bone according to embodiment 3, wherein, the force applied in step S3-2 is 170 kN with other process parameters remain unchanged.

Comparison 8

Prepare composite artificial bone according to embodiment 3, wherein, the force applied in step S3-2 is 170 kN and the pressure is kept for 10 seconds in step S3-3, with other process parameters remain unchanged.

Comparison 9

Prepare composite spinal fusion cage according to embodiment 4, wherein, the force applied in step S3-2 is 25 kN with other process parameters remain unchanged.

Comparison 10

Prepare composite spinal fusion cage according to embodiment 4, wherein, the force applied in step S3-2 is 225 kN with other process parameters remain unchanged.

Comparison 11

Prepare composite spinal fusion cage according to embodiment 4, wherein, the pressure is kept for 20 seconds in step S3-3 with other process parameters remain unchanged.

Comparison 12

Prepare composite artificial vertebral plate according to embodiment 5, wherein, the force applied in step S3-2 is 100 kN with other process parameters remain unchanged.

Comparison 13

Prepare composite artificial vertebral plate according to embodiment 5, wherein, the force applied in step S3-2 is 200 kN with other process parameters remain unchanged.

The mechanical properties of above comparisons were tested and the results are listed as follows:

| | | | Pressure | |
|---|---|---|---|---|
| | | | keeping | |
| No. | Pressure | | time | Mechanical properties |
| 1 | 133 | MPa | 90 s | Compressive strength is 32 MPa, and |
| | | | | bending strength is 21 MPa. |
| 2 | 1503 | MPa | 90 s | The sample layered and cracked when |
| | | | | demoulding. |
| 3 | 663 | MPa | 0 s | The sample cracked after demoulding. |
| 4 | 133 | MPa | 90 s | Compressive strength is 35 MPa, and |
| | | | | bending strength is 22 MPa. |
| 5 | 1503 | MPa | 90 s | The sample layered and cracked when |
| | | | | demoulding. |
| 6 | 88 | MPa | 90 s | The material crashed when demoulding. |
| 7 | 1503 | MPa | 90 s | The sample layered and cracked when |
| | | | | demoulding. |
| 8 | 1061 | MPa | 10 s | The sample cracked after demoulding. |
| 9 | 162 | MPa | 240 s | Compressive strength is 46 MPa, and |
| | | | | bending strength is 23 MPa. |
| 10 | 1461 | MPa | 240 s | The sample layered and cracked when |
| | | | | demoulding. |
| 11 | 779 | MPa | 20 s | The sample cracked after demoulding. |
| 12 | 74 | MPa | 270 s | The material crashed when demoulding. |
| 13 | 148 | MPa | 270 s | Compressive strength is 34 MPa, and |
| | | | | bending strength is 20 MPa. |

According to above comparisons, it can be concluded that:

when the force applied in step S3-2 was too small, the sample did not form (comparisons 6 and 11) or had a weak mechanical property that could not meet the requirement of bone repair at human load-bearing part (comparisons 1, 4, 8 and 12);

when the force applied in step S3-2 was too large, the sample layered, and then cracked while demoulding (comparisons 2, 5 and 9);

when the pressure keeping time in step S3-3 was too short (comparisons 8 and 11) or was even zero (comparison 3), although the force applied in step S3-2 made it possible to form a specific shape, the samples still cracked soon after demoulding due to the internal residual stress.

Comparison 14

Prepare bi-layer composite bone repair material, wherein, after covering collagen/nano-Ca-P/PLA mixture suspension obtained by step S2-2 onto the dense substrate in step S4-1, skip the standing process and directly proceed into freeze and freeze-drying in step S4-2. Other process parameters remain unchanged.

By testing, the dense layer and the porous layer separated for 6 of 10 samples prepared according to the comparison 14, and such separation also occurred for other samples in holding, moving and testing process. That was because in step S4-1 of above embodiment 4, the standing process could result in a slight dissolution on the upper surface on the dense layer and form an intermediate layer between the dense layer and the porous layer, thus integrating these two layers. Therefore, the standing process in step S4-1 is indispensable for preparing above said dense-porous bi-layer composite bone repair material.

1. A compression-resistant collagen-based artificial bone repair material, characterized in that the material is a dense and homogeneous organic/inorganic composite material, wherein,
the organic phase contains collagen,
the inorganic phase contains nano-sized calcium phosphate salt, and
the weight ratio of the organic phase to the inorganic phase is 9/1˜4/6.

2. A compression-resistant collagen-based artificial bone repair material according to claim 1, characterized in that the organic phase of the material further contains polyester, said polyester is one or more of poly (lactic acid), poly (glycolic acid), poly (lactic-co-glycolic acid), polycaprolactone and polydioxanone, the weight ratio of the organic phase to the inorganic phase is 9/1˜4/6, and the weight ratio of collagen to polyester is 9/1˜1/9.

3. A compression-resistant collagen-based artificial bone repair material according to claim 1 or 2, characterized in that the material possesses a compressive strength of 65˜150 MPa and a bending strength of 20˜100 MPa.

4. A compression-resistant collagen-based artificial bone repair material according to claim 1 or 2, characterized in that said nano-sized calcium phosphate salt possesses a particle size of 20˜200 nm, and a molar ratio of Ca/P=1/1˜2/1.

5. A compression-resistant collagen-based artificial bone repair material according to claim 1 or 2, characterized in that said nano-sized calcium phosphate salt is nano-sized hydroxyapatite with particle size of 20˜200 nm.

6. A compression-resistant collagen-based artificial bone repair material according to claim 2, characterized in that said polyester possesses a molecular weight of 50,000˜500,000.

7. A method for preparing compression-resistant collagen-based artificial bone repair material according to claim 1, characterized in that the method comprises following steps:
Step S1. Preparation of nano-Ca-P/collagen biomimetic composite powders, further comprising:
Step S1-1. Dissolve collagen in any one of hydrochloric acid, nitric acid or acetic acid to form an acidic collagen solution, wherein, the concentration of the collagen is 5.0×10⁻⁵˜5.0×10⁻³ g/mL;
Step S1-2. Keep stiffing the solution obtained by step S1-1 and add Ca²⁺ containing solution dropwise, wherein, the addition of Ca²⁺ is 0.01˜0.16 mol for 1 g of collagen;
Step S1-3. Keep stiffing the solution obtained by step S1-2 and add PO₄³⁻ containing solution dropwise, wherein, the molar ratio of the added PO₄³⁻ and the added Ca²⁺ in S1-2 is Ca/P=1/1˜2/1;
Step S1-4. Keep stirring the solution obtained by step S1-3 and add NaOH solution until the pH of the mixture system gets to 6˜8, wherein, precipitation appears when the pH of the mixture system gets to 5˜6, and white suspension will be obtained when the pH gets to 7;
Step S1-5. Stand the mixture system obtained by step S1-4 for 24˜120 hours, and then separate out the precipitation and wash it to remove impurity ions, followed by a freeze-drying, the composite powders will be obtained after gridding;
Step S2. Cold compression molding of the composite, further comprising:
Step S2-1. Weigh the composite powders obtained by step S1-5 and fill the powders into a cold compression dies;
Step S2-2. Compress the dies and make the pressure applied to the composite powders reaches 200˜1400 MPa;
Step S2-3. Keep the pressure for 30˜300 seconds, and then demould to obtain the Compression-resistant collagen-based artificial bone repair material.

8. A method for preparing Compression-resistant collagen-based artificial bone repair material according to claim 2, characterized in that the method comprises following steps:
Step S1. Preparation of nano-Ca-P/collagen biomimetic composite powders, further comprising:
Step S1-1. Dissolve collagen in any one of hydrochloric acid, nitric acid or acetic acid to form an acidic collagen solution, wherein, the concentration of the collagen is 5.0×10⁻⁵˜5.0×10⁻³ g/mL;
Step S1-2. Keep stiffing the solution obtained by step S1-1 and add Ca²⁺ containing solution dropwise, wherein, the addition of Ca²⁺ is 0.01˜0.16 mol for 1 g of collagen;
Step S1-3. Keep stiffing the solution obtained by step S1-2 and add PO₄³⁻ containing solution dropwise, wherein, the molar ratio of the added PO₄³⁻ and the added Ca²⁺ in S1-2 is Ca/P=1/1˜2/1;
Step S1-4. Keep stirring the solution obtained by step S1-3 and add NaOH solution until the pH of the mixture system gets to 6˜8, wherein, precipitation appears when the pH of the mixture system gets to 5˜6, and white suspension will be obtained when the pH gets to 7;
Step S1-5. Stand the mixture system obtained by step S1-4 for 24˜120 hours, and then separate out the precipitation and wash it to remove impurity ions, followed by a freeze-drying, the composite powders will be obtained after gridding;
Step S2. Preparation of collagen/nano-Ca-P/polyester composite, further comprising:
Step S2-1. Preparation polyester solution with a concentration of 0.02˜0.15 g/mL by dissolving polyester with molecular weight of 50,000˜200,000 into any one of 1,4-dioxane, dichloromethane, chloroform or dimethyl sulfoxide at 40˜70° C., said polyester is one or more of poly (lactic acid), poly (glycolic acid), poly (lactic-co-glycolic acid), polycaprolactone and polydioxanone;
Step S2-2. Add the composite powders obtained by step S1-5 into the polyester solution obtained by step S2-1 to form a collagen/nano-Ca-P/polyester mixture suspension system, wherein, the weight ratio of the composite powders to the polyester within the polyester solution is 1/2˜3/2;
Step S2-3. Put the mixture suspension system obtained by step S2-2 into an environment of −20˜4° C. to thoroughly freeze, and then freeze-dry for 24˜72 hours, followed by transferring to a vacuum drying oven to dry for 72˜120 hours, thus obtaining a collagen/nano-Ca-P/polyester composite;
Step S2-4. Smash the composite obtained by step S2-3 and sieve to screen out composite powders with particle size of 100˜600 μm;
Step S3. Cold compression molding of the composite, further comprising:
Step S3-1. Weigh the composite powders obtained by step S2-4 and fill the powders into a cold compression dies;
Step S3-2. Compress the dies and make the pressure applied to the composite powders reaches 200˜1400 MPa;
Step S3-3. Keep the pressure for 30˜300 seconds, and then demould to obtain the Compression-resistant collagen-based artificial bone repair material.

9. A method for preparing compression-resistant collagen-based artificial bone repair material according to claim 8, characterized in that in step S2-3, put the mixture suspension system obtained by step S2-2 into an environment of −20˜4° C. to thoroughly freeze, and then into liquid nitrogen to deep freeze, followed by freeze-dry for 24˜72 hours and vacuum dry for 72˜120 hours, thus obtaining said collagen/nano-Ca-P/polyester composite.

10. A compression-resistant collagen-based artificial bone repair material, characterized in that the material is a dense-porous bi-layer organic/inorganic composite, wherein,
said organic phase contains both collagen and polyester, said polyester is one or more of poly (lactic acid), poly (glycolic acid), poly (lactic-co-glycolic acid), polycaprolactone and polydioxanone,
the inorganic phase contains nano-sized calcium phosphate salt,
the weight ratio of the organic phase to the inorganic phase is 9/1˜2/8, and
the weight ratio of collagen to poly (lactic acid) is 9/1˜1/9;
said bi-layer structure contains:
a dense layer as the lower layer, with a thickness of 0.5˜5 mm, a compressive strength of 65˜150 MPa and a bending strength of 20˜100 MPa, and
a porous layer as the upper layer, with a thickness of 0.5˜5 mm and a porosity of 50%˜80%.

11. A compression-resistant collagen-based artificial bone repair material according to claim 9, characterized in that said nano-sized calcium phosphate salt possesses a particle size of 20˜200 nm, and a molar ratio of Ca/P=1/1˜2/1.

12. A compression-resistant collagen-based artificial bone repair material according to claim 9, characterized in that said nano-sized calcium phosphate salt is nano-sized hydroxyapatite with particle size of 20˜200 nm.

13. A compression-resistant collagen-based artificial bone repair material according to claim 9, characterized in that said polyester possesses a molecular weight of 50,000˜500,000.

14. A method for preparing compression-resistant collagen-based artificial bone repair material according to any one of claims 10-13, the method comprises following steps:
Step S1. Preparation of nano-Ca-P/collagen biomimetic composite powders, further comprising:
Step S1-1. Dissolve collagen in any one of hydrochloric acid, nitric acid or acetic acid to form an acidic collagen solution, wherein, the concentration of the collagen is 5.0×10⁻⁵˜5.0×10⁻³ g/mL;
Step S1-2. Keep stiffing the solution obtained by step S1-1 and add Ca²⁺ containing solution dropwise, wherein, the addition of Ca²⁺ is 0.01˜0.16 mol for 1 g of collagen;
Step S1-3. Keep stiffing the solution obtained by step S1-2 and add PO₄³⁻ containing solution dropwise, wherein, the molar ratio of the added PO₄³⁻ and the added Ca²⁺ in S1-2 is Ca/P=1/1˜2/1;
Step S1-4. Keep stirring the solution obtained by step S1-3 and add NaOH solution until the pH of the mixture system gets to 6˜8, wherein, precipitation appears when the pH of the mixture system gets to 5˜6, and white suspension will be obtained when the pH gets to 7;
Step S1-5. Stand the mixture system obtained by step S1-4 for 24˜120 hours, and then separate out the precipitation and wash it to remove impurity ions, followed by a freeze-drying, the composite powders will be obtained after gridding;
Step S2. Preparation of collagen/nano-Ca-P/polyester composite, further comprising:
Step S2-1. Preparation polyester solution with a concentration of 0.02˜0.15 g/mL by dissolving polyester with molecular weight of 50,000˜200,000 into any one of 1,4-dioxane, dichloromethane, chloroform or dimethyl sulfoxide at 40˜70° C., said polyester is one or more of poly (lactic acid), poly (glycolic acid), poly (lactic-co-glycolic acid), polycaprolactone and polydioxanone;
Step S2-2. Add the composite powders obtained by step S1-5 into the polyester solution obtained by step S2-1 to form a collagen/nano-Ca-P/polyester mixture suspension system, wherein, the weight ratio of the composite powders to the polyester within the polyester solution is 1/2˜3/2;
Step S2-3. Put the mixture suspension system obtained by step S2-2 into an environment of −20˜4° C. to thoroughly freeze, and then freeze-dry for 24˜72 hours, followed by transferring to a vacuum drying oven to dry for 72˜120 hours, thus obtaining a collagen/nano-Ca-P/polyester composite;
Step S2-4. Smash the composite obtained by step S2-3 and sieve to screen out composite powders with particle size of 100˜600 μm;
Step S3. Cold compression molding of the composite, further comprising:
Step S3-1. Weigh the composite powders obtained by step S2-4 and fill the powders into a cold compression dies;
Step S3-2. Compress the dies and make the pressure applied to the composite powders reaches 200˜1400 MPa;
Step S3-3. Keep the pressure for 30˜300 seconds, and then demould to obtain said dense layer;
Step S4. Fabrication of porous layer on the dense layer, further comprising:
Step S4-1. Use the dense layer obtained by step S3-3 as the substrate, and repeat steps S1-1˜S2-2 to cover a collagen/nano-Ca-P/polyester mixture suspension obtained by step S2-2 on such substrate, then standing for 2˜15 min, meanwhile slight solvation occurs on the substrate upper surface;
Step S4-2. Put the dense and the covered mixture suspension obtained by step S4-1 into a low-temperature environment of −20˜−10° C. to achieve quick-freezing, freeze-dry them for 24˜72 hours, and then transfer to a vacuum drying oven to dry for 72˜120 hours, thus finally obtain said dense-porous bi-layer Compression-resistant collagen-based artificial bone repair material.

15. A method for preparing compression-resistant collagen-based artificial bone repair material according to claim 14, characterized in that in step S2-3, put the mixture suspension system obtained by step S2-2 into an environment of −20˜4° C. to thoroughly freeze, and then into liquid nitrogen to deep freeze, followed by freeze-dry for 24˜72 hours and vacuum dry for 72˜120 hours, thus obtaining said collagen/nano-Ca-P/polyester composite.
